(19) United States
(12) Patent Application Publication
(10) Pub. No.: US 2019/0150828 A1
(43) Pub. Date: May 23, 2019

(54) WEARABLE DEVICE CAPABLE OF RECOGNIZING SLEEP STAGE AND RECOGNITION METHOD THEREOF

(71) Applicant: Kinpo Electronics, Inc., New Taipei City (TW)

(72) Inventors: Koichi Haraikawa, New Taipei City (TW); Jen-Chien Chien, New Taipei City (TW); Yin-Tsong Lin, New Taipei City (TW); Tsui-Shan Hung, New Taipei City (TW); Chien-Hung Lin, New Taipei City (TW)

(73) Assignee: Kinpo Electronics, Inc., New Taipei City (TW)

(21) Appl. No.: 15/990,838

(22) Filed: May 29, 2018

(30) Foreign Application Priority Data
Nov. 20, 2017 (TW) 106140053

(51) Int. Cl. A61B 5/00; A61B 5/0456; G06N 3/08; A61B 5/0245

(52) U.S. Cl. CPC A61B 5/4812; A61B 5/6801; A61B 5/0245; G06N 3/08; A61B 5/0456

(57) ABSTRACT

A wearable device capable of recognizing sleep stage including a processor, an electrocardiogram sensor, an acceleration sensor and an angular acceleration sensor is provided. The processor trains a neural network module. The electrocardiogram sensor generates an electrocardiogram signal. The processor analyzes the electrocardiogram signal to generate a plurality of first characteristic values. The acceleration sensor generates an acceleration signal. The processor analyzes the acceleration signal to generate a plurality of second characteristic values. The angular acceleration sensor generates an angular acceleration signal. The processor analyzes the angular acceleration signal to generate a plurality of third characteristic values. The processor utilizes the trained neural network module to perform a sleep stage recognition operation according to the first characteristic values, the second characteristic values and the third characteristic values, so as to obtain a sleep stage recognition result.

### CROSS-REFERENCE TO RELATED APPLICATION

This application claims the priority benefit of Taiwan application serial no. 106140053, filed on Nov. 20, 2017. The entirety of the above-mentioned patent application is hereby incorporated by reference herein and made a part of this specification.

### TECHNICAL FIELD

The disclosure relates to a wearable device, and more particularly, to a wearable device capable of recognizing sleep stage and a recognition method thereof.

### BACKGROUND

In the technical field for recognizing sleep stage, the common sleep stage recognition is usually conducted by assessment with a polysomnography (PSG) system through multiple physiological sensing operations. However, the physiological sensing operations include, for example, electroencephalography (EEG), electrooculography (EOG), electrocardiogram (ECG), electromyography (EMG), respiratory Effort, air flow, blood pressure, blood oxygen saturation (SaO₂), and heart rate and sleep gesture. In other words, the common sleep stage recognition requires complicated accessories to be worn by a wearer and requires analysis on a large amount of sense data. In consideration of the above, providing a wearable device capable of effectively sensing a sleep stage of the wearer having characteristics of convenience is one of important issues to be addressed in the field.

### SUMMARY

The disclosure provides a wearable device capable of recognizing sleep stage and a method thereof, which can effectively recognize the sleep stage of the wearer and provide characteristics of convenience.

A wearable device capable of recognizing sleep stage of the disclosure includes a processor, an electrocardiogram sensor, an acceleration sensor and an angular acceleration sensor. The processor is configured to train a neural network module. The electrocardiogram sensor is coupled to the processor. The electrocardiogram sensor generates an electrocardiogram signal. The processor analyzes the electrocardiogram signal to generate a plurality of first characteristic values. The acceleration sensor generates an acceleration signal. The processor analyzes the acceleration signal to generate a plurality of second characteristic values. The angular acceleration sensor generates an angular acceleration signal. The processor analyzes the angular acceleration signal to generate a plurality of third characteristic values. The processor utilizes the trained neural network module to perform a sleep stage recognition operation according to the first characteristic values, the second characteristic values and the third characteristic values, so as to obtain a sleep stage recognition result.

A recognition method of sleep stage is adapted to a wearable device. The wearable device includes a processor, an electrocardiogram sensor, an acceleration sensor and an angular acceleration sensor. The method includes: training a neural network module by the processor; generating an electrocardiogram signal by the electrocardiogram sensor, and analyzing the electrocardiogram signal by the processor to generate a plurality of first characteristic values; generating an acceleration signal by the acceleration sensor, and analyzing the acceleration signal by the processor to generate a plurality of second characteristic values; generating an angular acceleration signal by the angular acceleration sensor, and analyzing the angular acceleration signal by the processor to generate a plurality of third characteristic values; and utilizing the trained neural network module by the processor to perform a sleep stage recognition operation according to the first characteristic values, the second characteristic values and the third characteristic values, so as to obtain a sleep stage recognition result.

Based on the above, the wearable device capable of recognizing sleep stage and the method thereof according to the disclosure can sense a plurality of characteristic values by the electrocardiogram sensor, the acceleration sensor and the angular acceleration sensor. In this way, the processor can utilize the trained neural network module to perform the sleep stage recognition operation according to the characteristic values. As a result, the wearable device of the disclosure can effectively obtain a recognition result of the sleep stage and provide characteristics of convenience.

To make the above features and advantages of the disclosure more comprehensible, several embodiments accompanied with drawings are described in detail as follows.

### BRIEF DESCRIPTION OF THE DRAWINGS

The accompanying drawings are included to provide a further understanding of the disclosure, and are incorporated in and constitute a part of this specification. The drawings illustrate embodiments of the disclosure and, together with the description, serve to explain the principles of the disclosure.

FIG. 1 illustrates a block diagram of a wearable device in an embodiment of the disclosure.

FIG. 2 illustrates a waveform diagram of an electrocardiogram signal in an embodiment of the disclosure.

FIG. 3 illustrates a block diagram for analyzing the electrocardiogram signal in an embodiment of the disclosure.

FIG. 4 illustrates a waveform diagram of a plurality of first characteristic values in an embodiment of the disclosure.

FIG. 5 illustrates a waveform diagram of another plurality of first characteristic values in an embodiment of the disclosure.

FIG. 6 illustrates a block diagram for analyzing an acceleration signal in an embodiment of the disclosure.

FIG. 7 illustrates a waveform diagram of a plurality of second characteristic values in an embodiment of the disclosure.

FIG. 8 illustrates a block diagram for analyzing an angular acceleration signal in an embodiment of the disclosure.

FIG. 9 illustrates a flowchart of a recognition method of sleep stage in an embodiment of the disclosure.

### DETAILED DESCRIPTION

In the following detailed description, for purposes of explanation, numerous specific details are set forth in order to provide a thorough understanding of the disclosed embodiments. It will be apparent, however, that one or more embodiments may be practiced without these specific details. In other instances, well-known structures and devices are schematically shown in order to simplify the drawing.

In order to make content of the disclosure more comprehensible, embodiments are provided below to describe the disclosure in detail, however, the disclosure is not limited to the provided embodiments, and the provided embodiments can be suitably combined. Moreover, elements/components/steps with same reference numerals represent same or similar parts in the drawings and embodiments.

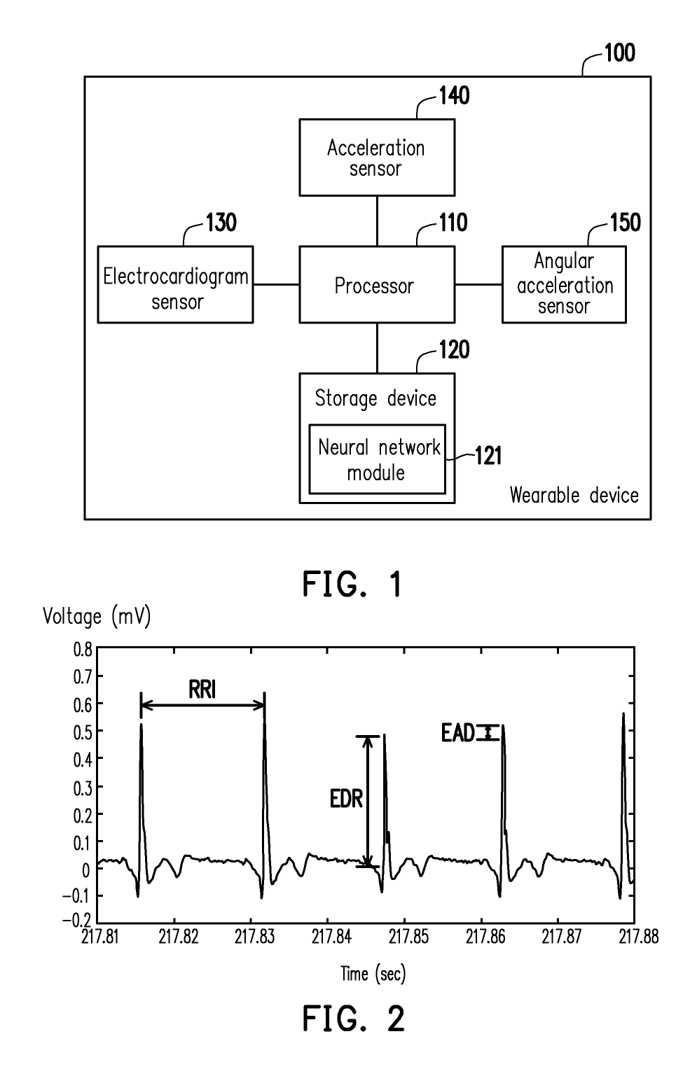

FIG. 1 illustrates a block diagram of a wearable device in an embodiment of the disclosure. With reference to FIG. 1, a wearable device 100 includes a processor 110, a storage device 120, an electrocardiogram sensor 130, an acceleration sensor 140 and an angular acceleration sensor 150. The processor 110 is coupled to the storage device 120, the electrocardiogram sensor 130, the acceleration sensor 140 and the angular acceleration sensor 150. In the present embodiment, the wearable device 100 may be, for example, smart clothes, smart wristbands or other similar devices, and the wearable device 100 is configured to recognize the sleep stage of the wearer. The wearable device 100 can integrate each of the sensors into one wearable item instead of complicated accessories. In other words, when the wearer is asleep, the sleep stage of the wearer may be sensed by the wearable device 100 worn by the wearer. In the present embodiment, a sleep recognition may be conducted for the wearer in a lying or sitting posture, for example, so as to monitor a sleep quality of the wearer and record a sleep situation. Further, in the present embodiment, the sleep stage recognizable by the wearable device 100 can include a wakefulness stage (Stage W), a first non-rapid eye movement stage (Stage N1, a.k.a. falling-asleep stage), a second non-rapid eye movement stage (Stage N2, a.k.a. slightly-deeper sleep stage), a third non-rapid eye movement stage (Stage N3, a.k.a. deep sleep stage) and a rapid eye movement (Stage R) according to Polysomnography (PSG). It should be noted that, a usage scenario of the wearable device 100 of the disclosure refers to a usage scenario in which the wearer is in the sleep situation where the deep sleep stage may take place in a longer period of time.

In the present embodiment, the electrocardiogram sensor 130 is configured to sense an electrocardiogram signal ECG and provide the electrocardiogram signal to the processor 110. The processor 110 analyzes the electrocardiogram signal to generate a plurality of first characteristic values. In the present embodiment, the acceleration sensor 140 is configured to sense an acceleration caused by moving of the wearer's body or wearing part and provide an acceleration signal to the processor 110. The processor 110 analyzes the acceleration signal to generate a plurality of second characteristic values. In the present embodiment, the angular acceleration sensor 150 is configured to sense an angular acceleration caused by turning of the wearer's body or wearing part and provide an angular acceleration signal to the processor 110. The processor 110 analyzes the angular acceleration signal to generate a plurality of third characteristic values. In other words, when the wearer is asleep, because the wearer may intentionally or unintentionally turn over or move his/her body, the wearable device 100 of the present embodiment can also sense the effect of turning or moving of the wearing part of the wearer in addition to sensing the electrocardiogram information of the wearer, such an analysis result provided by the processor 110 can take the effect caused by actions of the wearer in consideration.

In the present embodiment, the processor 110 is, for example, a central processing unit (CPU), a system on chi (SOC) or other programmable devices for general purpose or special purpose, such as a microprocessor and a digital signal processor (DSP), a programmable controller, an application specific integrated circuit (ASIC), a programmable logic device (PLD) or other similar devices or a combination of above-mentioned devices.

In the present embodiment, the storage device 120 is, for example, a dynamic random access memory (DRAM), a flash memory or a non-volatile random access memory (NVRAM). In the present embodiment, the storage device 120 is configured to store data and program modules described in each embodiment of the disclosure, which can be read and executed by the processor 110 so the wearable device 100 can realize the recognition method of sleep stage described in each embodiment of the disclosure.

In the present embodiment, the storage device 120 further includes a neural network module 121. The processor 110 can sense a plurality of characteristic values from different wearers in advance by the electrocardiogram sensor 130, the acceleration sensor 140 and the angular acceleration sensor 150, and use the characteristic values from the different wearers as sample data. In the present embodiment, the processor 110 can create a prediction model according to determination conditions, algorithms and parameters from the sleep stage, and use a plurality of the sample data for training or correcting the prediction model. Accordingly, when a sleep stage recognition operation is performed by the wearable device 100 for the wearer, the processor 110 can utilize the trained neural network module 121 to obtain a recognition result according to the first characteristic values, the second characteristic values and the third characteristic values sensed by the electrocardiogram sensor 130, the acceleration sensor 140 and the angular acceleration sensor 150. Nevertheless, enough teaching, suggestion, and implementation illustration regarding algorithms and calculation modes for the trained neural network module 121 of the present embodiment may be obtained with reference to common knowledge in the related art, which is not repeated hereinafter.

FIG. 2 illustrates a waveform diagram of an electrocardiogram signal in an embodiment of the disclosure. With reference to FIG. 1 and FIG. 2, the electrocardiogram signal sensed by the electrocardiogram sensor 130 is as shown in FIG. 2. In the present embodiment, the processor 110 can perform a heart rate variability (HRV) analysis operation to analyze a heart beat interval variation (R-R interval) RRI of the electrocardiogram signal, so as to obtain a plurality R-wave signals in the electrocardiogram signal. The processor 110 can perform the heart rate variability analysis operation to analyze variation of the R-wave signals. Further, the processor 110 can also perform a R-wave amplitude analysis operation and an adjacent R-waves difference (Difference of Amplitude between R and next R, EAD) analysis operation to analyze the R-wave signals. For instance, in the present embodiment, a distance between two R-waves may be used as the heart beat interval variation RRI. The R-wave amplitude analysis operation is, for example, to analyze a R-wave amplitude EDR in the electrocardiogram in order to obtain an ECG-derived respiratory signal, wherein peak and trough of the R-wave may be used as the R-wave amplitude EDR. A difference between the peaks of two adjacent R-waves may be used as the adjacent R-waves difference.

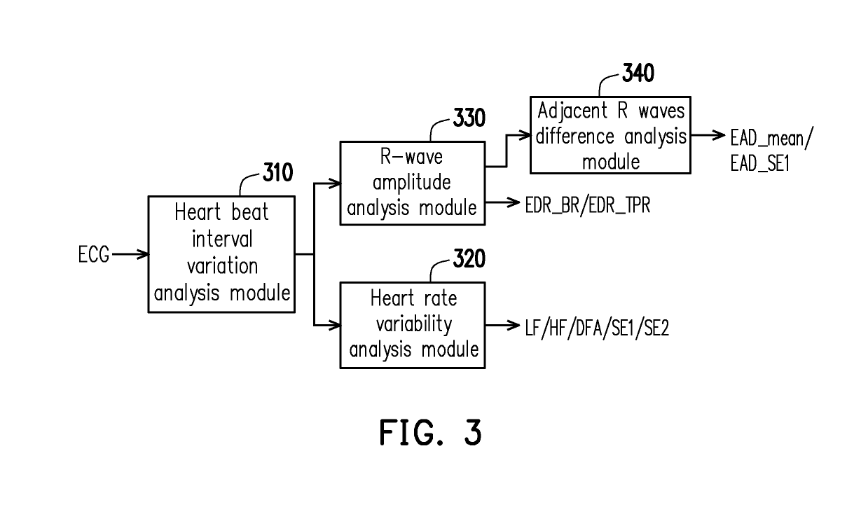

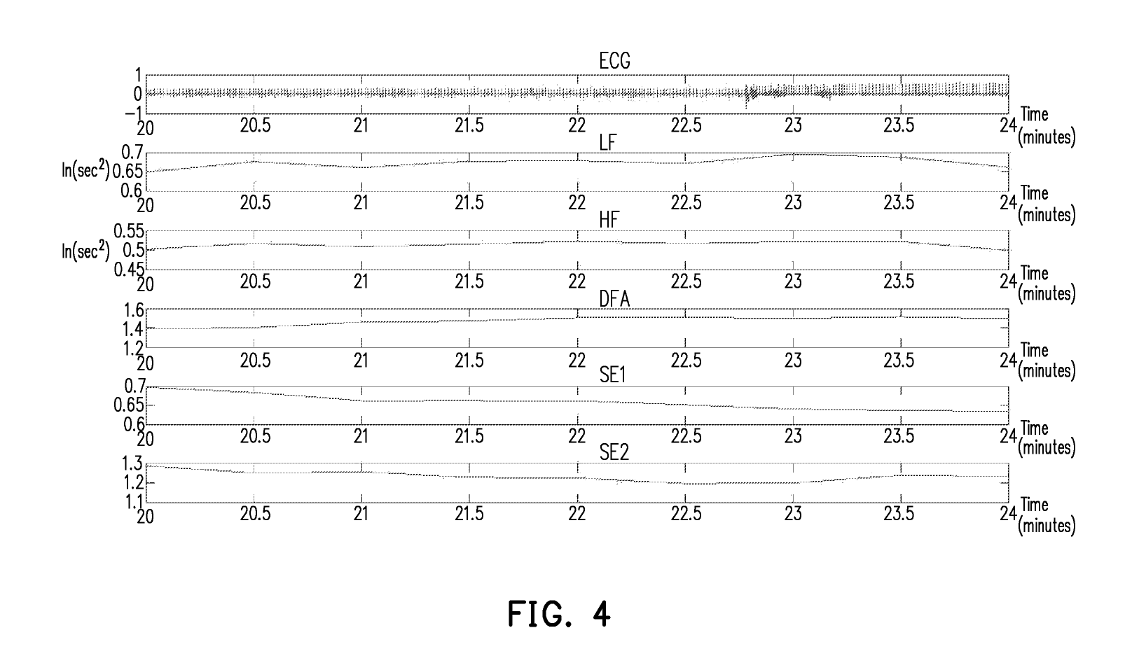

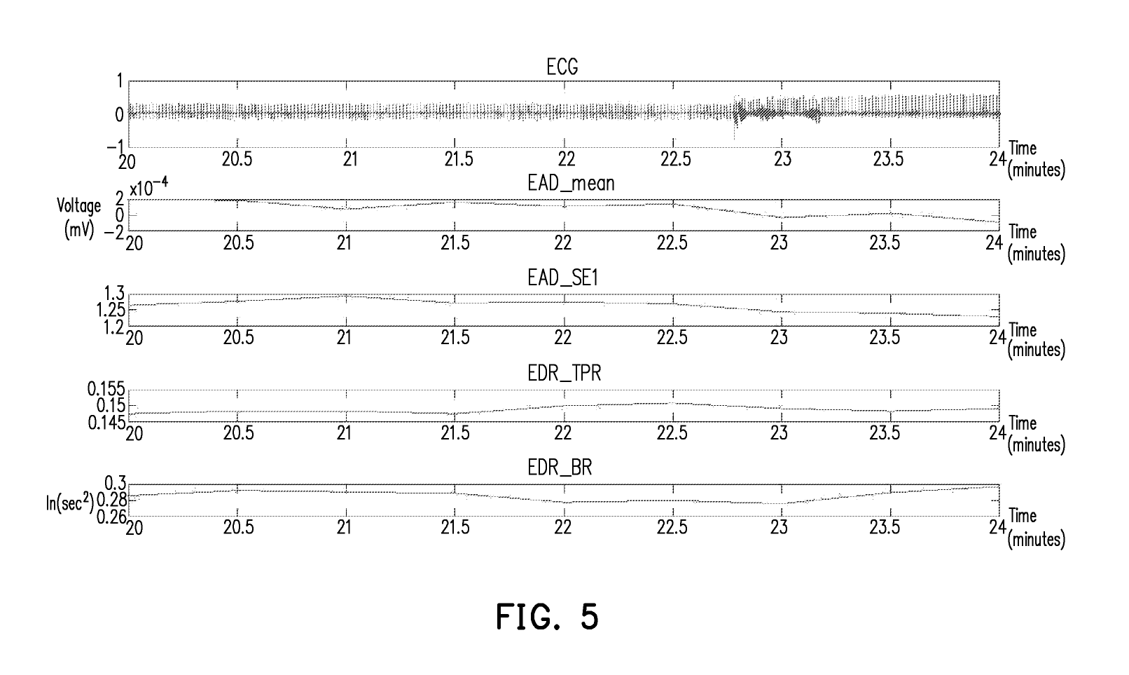

FIG. 3 illustrates a block diagram for analyzing the electrocardiogram signal in an embodiment of the disclosure. FIG. 4 illustrates a waveform diagram of a plurality of first characteristic values in an embodiment of the disclosure. FIG. 5 illustrates a waveform diagram of another plurality of first characteristic values in an embodiment of the disclosure. It should be noted that, each of the following waveform diagrams shows, for example, a sleep recognition operation performed per 30 seconds within a time length of 5 minutes. With reference to FIG. 1 to FIG. 5, the storage device 120 can store, for example, a heart beat interval variation analysis module 310, a heart rate variability analysis module 320, a R-wave amplitude analysis module 330 and an adjacent R-waves difference analysis module 340. In the present embodiment, the processor 110 receives the electrocardiogram signal ECG of the wearer provided by the electrocardiogram sensor, and analyzes the electrocardiogram signal ECG by the heart beat interval variation analysis module 310, so as to obtain a plurality of R-wave signal as shown in FIG. 2.

In the present embodiment, the heart rate variability analysis module 320 analyzes the R-wave signals, so as to obtain a low frequency signal LF, a high frequency signal HF, a detrended fluctuation analysis signal DFA, a first sample entropy signal SE1 and a second sample entropy signal SE2 as shown in FIG. 4. In the present embodiment, the low frequency signal LF is, for example, a signal with strength ranged from 0.04 Hz to 0.15 Hz among the R-wave signals. The high frequency signal HF is, for example, a signal with strength ranged from 0.15 Hz to 0.4 Hz among the R-wave signals. The detrended fluctuation analysis signal DFA is, for example, a signal underwent a detrended fluctuation analysis (DFA) among the R-wave signals. The first sample entropy signal SE1 is, for example, a signal underwent a sample entropy operation with the number of samples being 1 among the R-wave signals. The second sample entropy signal SE2 is, for example, a signal underwent a sample entropy operation with the number of samples being 2 among the R-wave signals.

In the present embodiment, the R-wave amplitude analysis module 330 analyzes the R-wave signals, so as to obtain a result including a turning point ratio value EDR_TPR and a signal strength value EDR_BR as shown in FIG. 5. Further, in the present embodiment, the adjacent R-waves difference analysis module 340 analyzes the R-wave signals, so as to obtain a mean value EAD_mean and a sample entropy value EAD_SE1 as shown in FIG. 5. In other words, the first characteristic values of the present embodiment are obtained from the low frequency signal LF, the high frequency signal HF, the detrended fluctuation analysis signal DFA, the first sample entropy signal SE1 and the second sample entropy signal SE2, and include the turning point ratio value EDR_TPR, the signal strength value EDR_BR, mean value EAD_mean and a sample entropy value EAD_SE1.

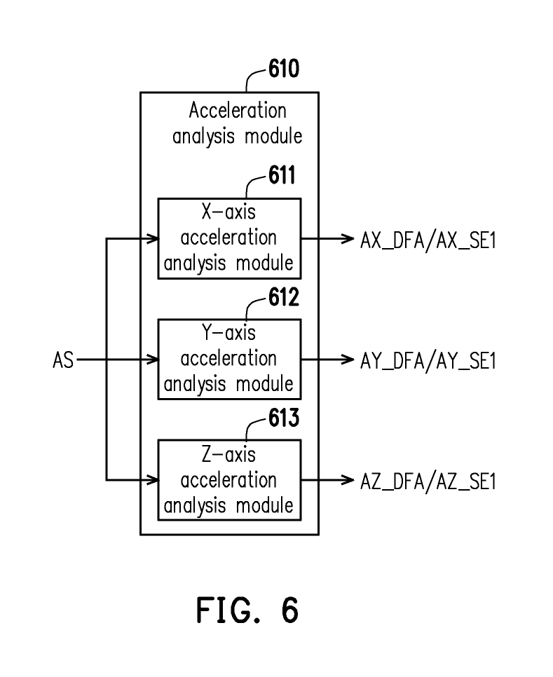

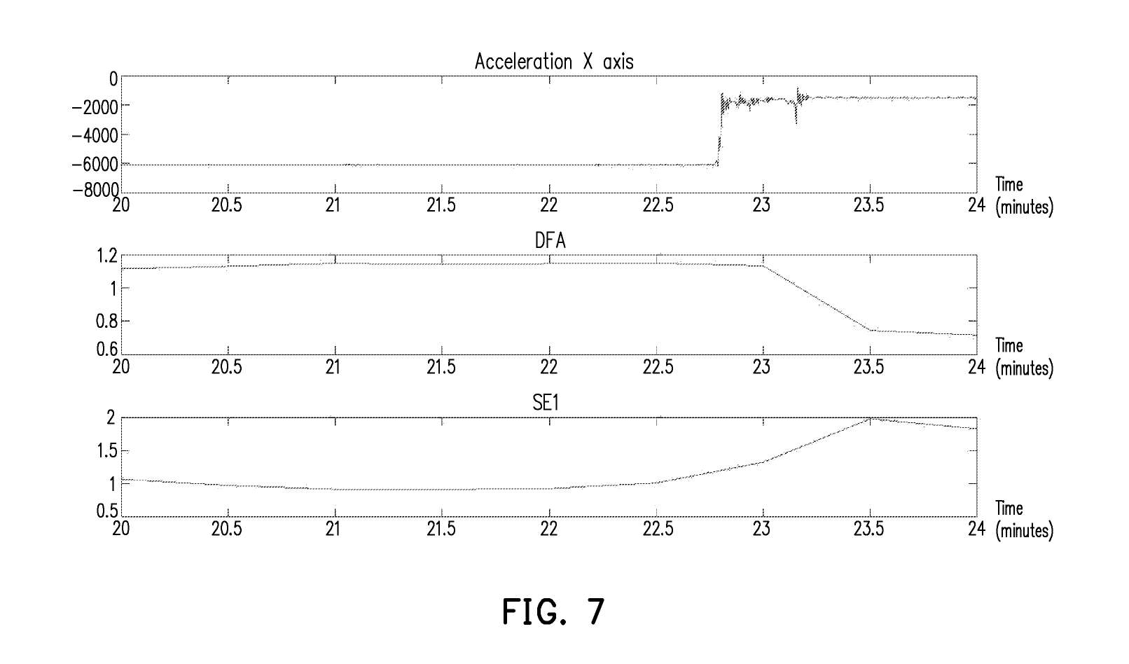

FIG. 6 illustrates a block diagram for analyzing an acceleration signal in an embodiment of the disclosure. FIG. 7 illustrates a waveform diagram of a plurality of second characteristic values in an embodiment of the disclosure. With reference to FIG. 1, FIG. 6 and FIG. 7, the storage device 120 can store, for example, an acceleration analysis module 610. In the present embodiment, the acceleration analysis module 610 includes an X-axis acceleration analysis module 611, a Y-axis acceleration analysis module 612 and a Z-axis acceleration analysis module 613. The acceleration sensor 140 can sense an acceleration signal AS of the wearer, wherein the acceleration sensor 140 may be, for example, a gravity sensor (G sensor). The acceleration analysis module 610 can analyze the acceleration signal AS to obtain respective acceleration values of X-axis (a first direction axis), Y-axis (a second direction axis) and Z-axis (a third direction axis), and obtain respective acceleration detrended fluctuation analysis values and respective acceleration sample entropy values by the X-axis acceleration analysis module 611, the Y-axis acceleration analysis module 612 and the Z-axis acceleration analysis module 613. For instance, the X-axis acceleration analysis module 611, the Y-axis acceleration analysis module 612 and the Z-axis acceleration analysis module 613 can obtain an acceleration detrended fluctuation analysis value DFA and an acceleration sample entropy value SE1 as shown in FIG. 7.

In the present embodiment, the X-axis acceleration analysis module 611 can generate a first acceleration detrended fluctuation analysis value AX_DFA and a first acceleration sample entropy value AX_SE1. The Y-axis acceleration analysis module 612 can generate a second acceleration detrended fluctuation analysis value AY_DFA and a second acceleration sample entropy value AY_SE2. The Z-axis acceleration analysis module 613 can generate a third acceleration detrended fluctuation analysis value AZ_DFA and a third acceleration sample entropy value AZ_SE3. In other words, the second characteristic values of the present embodiment include the first acceleration detrended fluctuation analysis value AX_DFA, the first acceleration sample entropy value AX_SE1, the second acceleration detrended fluctuation analysis value AY_DFA, the second acceleration sample entropy value AY_SE2, the third acceleration detrended fluctuation analysis value AZ_DFA and the third acceleration sample entropy value AZ_SE3.

Nevertheless, enough teaching, suggestion, and implementation illustration regarding details of the related calculations for the acceleration detrended fluctuation analysis values and the acceleration sample entropy values of the present embodiment may be obtained with reference to common knowledge in the related art, which is not repeated hereinafter.

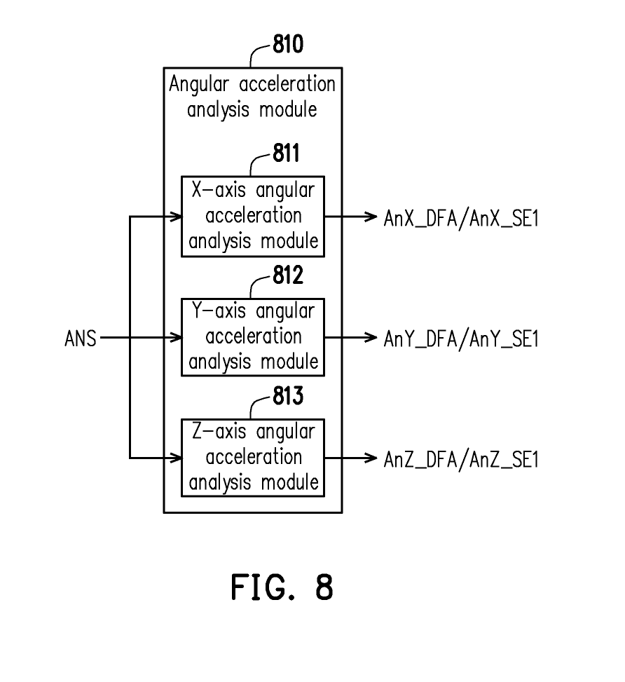

FIG. 8 illustrates a block diagram for analyzing an angular acceleration signal in an embodiment of the disclosure. With reference to FIG. 1 and FIG. 8, the storage device 120 can store, for example, an angular acceleration analysis module 810. In the present embodiment, the angular acceleration analysis module 810 includes an X-axis angular acceleration analysis module 811, a Y-axis angular acceleration analysis module 812 and a Z-axis angular acceleration analysis module 813. The angular acceleration sensor 150 can sense an angular acceleration signal AS of the wearer, wherein the angular acceleration sensor 150 may be, for example, a gyroscope. The angular acceleration analysis module 810 can analyze the angular acceleration signal AS to obtain respective angular acceleration values of X-axis (a first direction axis), Y-axis (a second direction axis) and Z-axis (a third direction axis), and obtain respective angular acceleration detrended fluctuation analysis values and respective angular acceleration sample entropy values by the X-axis angular acceleration analysis module 811, the Y-axis angular acceleration analysis module 812 and the Z-axis angular acceleration analysis module 813.

In the present embodiment, the X-axis angular acceleration analysis module 811 can generate a first angular acceleration detrended fluctuation analysis value AnY_DFA and a first angular acceleration sample entropy value AnX_SE1. The Y-axis angular acceleration analysis module 812 can generate a second angular acceleration detrended fluctuation analysis value AnY_DFA and a second angular acceleration sample entropy value AnY_SE2. The Z-axis angular acceleration analysis module 813 can generate a third angular acceleration detrended fluctuation analysis value AnZ_DFA and a third angular acceleration sample entropy value AnZ_SE3. In other words, the third characteristic values of the present embodiment include the first angular acceleration detrended fluctuation analysis value AnX_DFA, the first angular acceleration sample entropy value AnX_SE1, the second angular acceleration detrended fluctuation analysis value AnY_DFA, the second angular acceleration sample entropy value AnY_SE2, the third angular acceleration detrended fluctuation analysis value AnZ_DFA and the third angular acceleration sample entropy value AnZ_SE3.

Nevertheless, enough teaching, suggestion, and implementation illustration regarding details of the related calculations for the angular acceleration detrended fluctuation analysis values and the angular acceleration sample entropy values of the present embodiment may be obtained with reference to common knowledge in the related art, which is not repeated hereinafter.

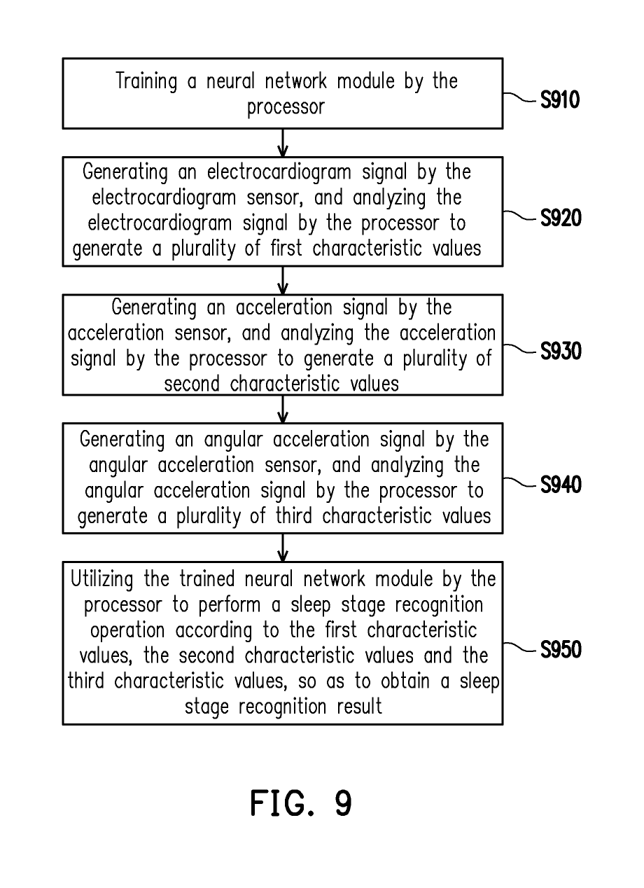

FIG. 9 illustrates a flowchart of a recognition method of sleep stage in an embodiment of the disclosure. With reference to FIG. 1 and FIG. 9, the recognition method of the present embodiment is at least adapted to the wearable device 100 of FIG. 1. In step S910, the processor 110 trains the neural network module 121. In step S920, the electrocardiogram sensor 130 generates an electrocardiogram signal, and the processor 110 analyzes the electrocardiogram signal to generate a plurality of first characteristic values. In step S930, the acceleration sensor 140 generates an acceleration signal, and the processor 110 analyzes the acceleration signal to generate a plurality of second characteristic values. In step S940, the angular acceleration sensor 150 generates an angular acceleration signal, and the processor 110 analyzes the angular acceleration signal to generate a plurality of third characteristic values. In step S950, the processor 110 utilizes the trained neural network module 121 to perform a sleep stage recognition operation according to the first characteristic values, the second characteristic values and the third characteristic values, so as to obtain a sleep stage recognition result.

In this way, the recognition method of the present embodiment can recognize the sleep stage of the wearer according to said 21 characteristic values sensed by the electrocardiogram sensor 130, the acceleration sensor 140 and the angular acceleration sensor 150. In the present embodiment, the sleep stage recognition result is one of the wakefulness stage, the first non-rapid eye movement stage, the second non-rapid eye movement stage, the third non-rapid eye movement stage and the rapid eye movement stage. Also, the wakefulness stage, the first non-rapid eye movement stage, the second non-rapid eye movement stage, the third non-rapid eye movement stage and the rapid eye movement stage are established by a polysomnography standard.

In addition, sufficient teaching, suggestion, and implementation regarding detailed features of each module and each sensor in the wearable device 100 of the present embodiment the disclosure may be obtained from the foregoing embodiments of FIG. 1 to FIG. 8, and thus related descriptions thereof are not repeated hereinafter.

In summary, the wearable device capable of recognizing sleep stage and the recognition method of sleep stage according to the disclosure can provide an accurate sleep recognition function. The wearable device includes the electrocardiogram sensor, the acceleration sensor and the angular acceleration sensor so the wearable device can sense the electrocardiogram information and posture variation of the wearer. The wearable device of the disclosure can use the trained neural network module to perform the sleep stage recognition operation according to the characteristic values provided by the electrocardiogram sensor, the acceleration sensor and the angular acceleration sensor. Moreover, the wearable device of the disclosure can integrate each of the sensors into one wearable item instead of complicated accessories. As a result, the wearable device of the disclosure is suitable for the wearer to conveniently and effectively monitor the sleep state in a home environment so as to obtain the recognition result of the sleep stage of the wearer.

Although the present disclosure has been described with reference to the above embodiments, it will be apparent to one of ordinary skill in the art that modifications to the described embodiments may be made without departing from the spirit of the disclosure. Accordingly, the scope of the disclosure will be defined by the attached claims and not by the above detailed descriptions.

1. A wearable device capable of recognizing sleep stage, comprising:
a processor, configured to train a neural network module;
an electrocardiogram sensor, coupled to the processor, and configured to generate an electrocardiogram signal, wherein the processor analyzes the electrocardiogram signal to generate a plurality of first characteristic values;
an acceleration sensor, coupled to the processor, and configured to generate an acceleration signal, wherein the processor analyzes the acceleration signal to generate a plurality of second characteristic values; and
an angular acceleration sensor, coupled to the processor, and configured to generate an angular acceleration signal, wherein the processor analyzes the angular acceleration signal to generate a plurality of third characteristic values,
wherein the processor utilizes the trained neural network module to perform a sleep stage recognition operation according to the first characteristic values, the second characteristic values and the third characteristic values, so as to obtain a sleep stage recognition result.

2. The wearable device according to claim 1, wherein the processor performs a heart rate variability analysis operation to analyze a heart beat interval variation of the electrocardiogram signal, so as to obtain a low frequency signal, a high frequency signal, a detrended fluctuation analysis signal, a first sample entropy signal and a second sample entropy signal,
wherein the first characteristic values are obtained from the low frequency signal, the high frequency signal, the detrended fluctuation analysis signal, the first sample entropy signal and the second sample entropy signal.

3. The wearable device according to claim 1, wherein the processor performs a R-wave amplitude analysis operation to analyze a heart beat interval variation of the electrocardiogram signal, so as to obtain a turning point ratio value and a signal strength value,
wherein the first characteristic values comprise the turning point ratio value and the signal strength value.

4. The wearable device according to claim 3, wherein the processor performs an adjacent R-waves difference analysis operation to analyze the heart beat interval variation of the electrocardiogram signal, so as to obtain a mean value and a sample entropy value,
wherein the first characteristic values comprise the mean value and the sample entropy value.

5. The wearable device according to claim 1, wherein the processor obtains a first acceleration value corresponding to a first direction axis, a second acceleration value corresponding to a second direction axis and a third acceleration value corresponding to a third direction axis according to the acceleration signal,
wherein the processor analyzes the first acceleration value, the second acceleration value and the third acceleration value, so as to obtain a first acceleration detrended fluctuation analysis value, a first acceleration sample entropy value, a second acceleration detrended fluctuation analysis value, a second acceleration sample entropy value, a third acceleration detrended fluctuation analysis value and a third acceleration sample entropy value,
wherein the second characteristic values comprise the first acceleration detrended fluctuation analysis value, the first acceleration sample entropy value, the second acceleration detrended fluctuation analysis value, the second acceleration sample entropy value, the third acceleration detrended fluctuation analysis value and the third acceleration sample entropy value.

6. The wearable device according to claim 1, wherein the processor obtains a first angular acceleration value corresponding to a first direction axis, a second angular acceleration value corresponding to a second direction axis and a third angular acceleration value corresponding to a third direction axis according to the angular acceleration signal,
wherein the processor analyzes the first angular acceleration value, the second angular acceleration value and the third angular acceleration value, so as to obtain a first angular acceleration detrended fluctuation analysis value, a first angular acceleration sample entropy value, a second angular acceleration detrended fluctuation analysis value, a second angular acceleration sample entropy value, a third angular acceleration detrended fluctuation analysis value and a third angular acceleration sample entropy value,
wherein the third characteristic values comprise the first angular acceleration detrended fluctuation analysis value, the first angular acceleration sample entropy value, the second angular acceleration detrended fluctuation analysis value, the second angular acceleration sample entropy value, the third angular acceleration detrended fluctuation analysis value and the third angular acceleration sample entropy value.

7. The wearable device according to claim 1, wherein the sleep stage recognition result is one of a wakefulness stage, a first non-rapid eye movement stage, a second non-rapid eye movement stage, a third non-rapid eye movement stage and a rapid eye movement stage, and the wakefulness stage, the first non-rapid eye movement stage, the second non-rapid eye movement stage, the third non-rapid eye movement stage and the rapid eye movement stage are established by a polysomnography standard.

8. The wearable device according to claim 1, wherein the processor pre-trains the neural network module according to a plurality of sample data, and each of the sample data comprises another plurality of first characteristic values, another plurality of second characteristic values and another plurality of third characteristic values.

9. A recognition method of sleep stage, adapted to a wearable device, the wearable device comprising a processor, an electrocardiogram sensor, an acceleration sensor and an angular acceleration sensor, the method comprising:
training a neural network module by the processor;
generating an electrocardiogram signal by the electrocardiogram sensor, and analyzing the electrocardiogram signal by the processor to generate a plurality of first characteristic values;
generating an acceleration signal by the acceleration sensor, and analyzing the acceleration signal by the processor to generate a plurality of second characteristic values;
generating an angular acceleration signal by the angular acceleration sensor, and analyzing the angular acceleration signal by the processor to generate a plurality of third characteristic values; and
utilizing the trained neural network module by the processor to perform a sleep stage recognition operation according to the first characteristic values, the second characteristic values and the third characteristic values, so as to obtain a sleep stage recognition result.

10. The recognition method of sleep stage according to claim 9, wherein the step of analyzing the electrocardiogram signal by the processor to generate the first characteristic values comprises:
performing a heart rate variability analysis operation by the processor to analyze a heart beat interval variation of the electrocardiogram signal, so as to obtain a low frequency signal, a high frequency signal, a detrended fluctuation analysis signal, a first sample entropy signal and a second sample entropy signal,
wherein the first characteristic values are obtained from the low frequency signal, the high frequency signal, the detrended fluctuation analysis signal, the first sample entropy signal and the second sample entropy signal.

11. The recognition method of sleep stage according to claim 9, wherein the step of analyzing the electrocardiogram signal by the processor to generate the first characteristic values comprises:
performing a R-wave amplitude analysis operation by the processor to analyze a heart beat interval variation of the electrocardiogram signal, so as to obtain a turning point ratio value and a signal strength value,
wherein the first characteristic values comprise the turning point ratio value and the signal strength value.

12. The recognition method of sleep stage according to claim 11, further comprising:
performing an adjacent R-waves difference analysis operation by the processor to analyze the heart beat interval variation of the electrocardiogram signal, so as to obtain a mean value and a sample entropy value,
wherein the first characteristic values comprise the mean value and the sample entropy value.

13. The recognition method of sleep stage according to claim 9, wherein the step of analyzing the acceleration signal by the processor to generate the second characteristic values comprises:
obtaining a first acceleration value corresponding to a first direction axis, a second acceleration value corresponding to a second direction axis and a third acceleration value corresponding to a third direction axis by the processor according to the acceleration signal; and
analyzing the first acceleration value, the second acceleration value and the third acceleration value by the processor, so as to obtain a first acceleration detrended fluctuation analysis value, a first acceleration sample entropy value, a second acceleration detrended fluctuation analysis value, a second acceleration sample entropy value, a third acceleration detrended fluctuation analysis value and a third acceleration sample entropy value,
wherein the second characteristic values comprise the first acceleration detrended fluctuation analysis value, the first acceleration sample entropy value, the second acceleration detrended fluctuation analysis value, the second acceleration sample entropy value, the third acceleration detrended fluctuation analysis value and the third acceleration sample entropy value.

14. The recognition method of sleep stage according to claim 9, wherein the step of analyzing the angular acceleration signal by the processor to generate the third characteristic values comprises:
obtaining a first angular acceleration value corresponding to a first direction axis, a second angular acceleration value corresponding to a second direction axis and a third angular acceleration value corresponding to a third direction axis by the processor according to the angular acceleration signal;
analyzing the first angular acceleration value, the second angular acceleration value and the third angular acceleration value by the processor, so as to obtain a first angular acceleration detrended fluctuation analysis value, a first angular acceleration sample entropy value, a second angular acceleration detrended fluctuation analysis value, a second angular acceleration sample entropy value, a third angular acceleration detrended fluctuation analysis value and a third angular acceleration sample entropy value,
wherein the third characteristic values comprise the first angular acceleration detrended fluctuation analysis value, the first angular acceleration sample entropy value, the second angular acceleration detrended fluctuation analysis value, the second angular acceleration sample entropy value, the third angular acceleration detrended fluctuation analysis value and the third angular acceleration sample entropy value.

15. The recognition method of sleep stage according to claim 9, wherein the sleep stage recognition result is one of a wakefulness stage, a first non-rapid eye movement stage, a second non-rapid eye movement stage, a third non-rapid eye movement stage and a rapid eye movement stage, and the wakefulness stage, the first non-rapid eye movement stage, the second non-rapid eye movement stage, the third non-rapid eye movement stage and the rapid eye movement stage are established by a polysomnography standard.

16. The recognition method of sleep stage according to claim 9, wherein the processor pre-trains the neural network module according to a plurality of sample data, and each of the sample data comprises another plurality of first characteristic values, another plurality of second characteristic values and another plurality of third characteristic values.
